(19) United States
(12) Patent Application Publication
(10) Pub. No.: US 2020/0051688 A1
(43) Pub. Date: Feb. 13, 2020

(54) ALERT SYSTEM

(71) Applicant: MY MEDIC WATCH PTY LTD, Sydney (AU)

(72) Inventors: Elizabeth BLANCHARD, Sydney (AU); Laurent PARSY, Sydney (AU); Bruce BREW, Sydney (AU); Helene BLANCHARD, Sydney (AU); Andreanne BLANCHARD, Sydney (AU); Serge LAURIOU, Sydney (AU)

(73) Assignee: My Medic Watch Ltd, Sydney (AU)

(21) Appl. No.: 16/339,220

(22) PCT Filed: Oct. 5, 2017
(86) PCT No.: PCT/AU2017/000209

§ 371 Date: Apr. 3, 2019

(30) Foreign Application Priority Data
Oct. 5, 2016 (AU) 2016904045

(51) Int. Cl. G16H 40/40; A61B 5/00; A61B 5/0205; G16H 40/63; G16H 40/67

(52) U.S. Cl. CPC G16H 40/40; A61B 5/0022; A61B 2562/0219; G16H 40/63; G16H 40/67; A61B 5/02055

(57) ABSTRACT

A system is provided which, in at least some embodiments, can read the vital signs of the body of a user utilising a sensing device such as a smartwatch or smart phone (for example utilising the iOS, Android or Pebble operating systems) and apply algorithms to interpret the vital signs and then send a notification with an escalation process to nominated carers if the patient is interpreted as having a fall or fit or seizure. In at least some embodiments doctors or other parties can log in to a secured dashboard and check a patient data in real time. Also in at least some preferred forms doctors or other parties can analyse the history of the patient. In at least some embodiments users/patients can also use data to keep track of fall or fit or seizure episodes and monitor their progress. Embodiments of the invention can be applied for example in situations where the patient/user suffers from a medical condition such as epilepsy and which may predispose the patient/user to falls and related events.

### TECHNICAL FIELD

The present invention relates to an alert system and, more particularly although not exclusively, to such a system adapted, although not exclusively, to assist in the management of people who may be prone to falling, whether by medical condition, age or otherwise.

### BACKGROUND

To date systems which monitor people have not been specifically adapted to detect selected conditions including one or more of specific conditions being a fall condition, a seizure or a sleepwalk event or related events, to systematically analyse the event and communicate the event both locally and to a remote location.

U.S. Pat. No. 9,689,887 assigned to Amazon Technologies describes a methodology for detecting a fall event associated with a parcel or the like.

However detection of a fall condition of a human body requires a different approach because of the complexity and variation of the manner in which a human may fall to the ground.

In particular forms the primary sensing will be carried out by a body worn sensor and more particularly a limb mounted sensor and more particularly a wrist mounted sensor. Again, there is complexity associated with using a limb to sense movement pertinent to the entire human body.

It is an object of the present invention to address or at least ameliorate some of the above disadvantages.

It will also be advantageous if the alert system can be adapted to sense, analyse and communicate other conditions instead of or in addition to the fall condition referenced above thereby to provide a multifunctional alert system.

### NOTES

The term “comprising” (and grammatical variations thereof) is used in this specification in the inclusive sense of “having” or “including”, and not in the exclusive sense of “consisting only of”.

The above discussion of the prior art in the Background of the invention, is not an admission that any information discussed therein is citable prior art or part of the common general knowledge of persons skilled in the art in any country.

### SUMMARY OF INVENTION

### Definitions:

In this specification a body worn sensor or wearable device sensor is a sensor which is mechanically associated with the body of a user such that the sensor can sense at least acceleration of the body relative to a reference frame. In particular forms the primary sensing for embodiments of the present invention will be carried out by a body worn sensor and more particularly a limb mounted sensor and more particularly a wrist mounted sensor.

In this specification a reference frame is a reference frame pertinent to sensing of acceleration of the body. In preferred instances the reference frame will be the surface upon which the user is supported. In most instances the reference frame will be the earth. In the case where the user is already moving with respect to the earth-for example where they are in a lift or an aeroplane or other moving vehicle then the reference frame will be that lift or aeroplane or vehicle and more particularly the surface within that vehicle or lift or aeroplane upon which the user is supported.

Accordingly in one broad form of the invention there is provided an alert system for communicating an event sensed by a body worn sensor.

Preferably the body worn sensor is mechanically associated with the body.

Preferably the event is a fall event.

Preferably the sensor includes a processor in communication with memory for on-board processing of at least one signal.

Preferably the sensor includes a timer.

Preferably the sensor includes a GPS device.

Preferably the sensor includes a communications device.

Preferably the communications device includes broadband network interconnectivity for connection to the Internet.

Preferably the communications device includes cellular telephone network interconnectivity for connection of the device to a local cellular telephone network.

Preferably the sensor includes an accelerometer.

Preferably the at least one signal is an acceleration signal.

Preferably the at least one signal is a timing signal.

Preferably the signal is an acceleration signal derived from the accelerometer.

Preferably the signal is a timing signal derived from the timer.

Preferably the signal is a GPS signal derived from the GPS device.

Preferably the event is a fall event.

Preferably the event is a seizure event.

Preferably the event is a sleepwalk event.

In a preferred form the system further includes an additional monitoring or sensing device.

Preferably the additional monitoring or sensing device includes at least a speaker and a microphone and is in communication with a web enabled server.

Preferably the web enabled server executes an application whereby functionality of the body worn sensor is supplemented with the functionality of the additional monitoring or sensing device.

Preferably the body worn sensor is mounted to the wrist of a user.

Preferably an artificial intelligence AI capability is programmed into memory 18 of the sensor for execution by processor 1 of the body worn sensor.

Preferably an AI program is executed on the processor associated with server located remote from the sensor 14.

Preferably the AI capability learns from false positive event determination and false negative event determination in order to statistically improve reliability of detection of an event over time and with particular reference to learned attributes of the data associated with any given user 12.

In a further broad form of the invention there is provided a fall detection apparatus comprising:

an accelerometer which communicates an acceleration signal to a processor;

the acceleration signal quantifying acceleration on a substantially continuous basis relative to a reference frame;

a timer which communicates a time reference signal to the processor;

the processor monitoring the acceleration signal on a substantially continuous basis;

the processor monitoring the timing signal on a substantially continuous basis;

and whereby if the acceleration signal is within a first low acceleration range for a predetermined period of time and is followed by a second high acceleration signal in a second predetermined period of time a fall condition is determined by the processor.

27. The fall detection apparatus of claim 26 wherein the processor monitors the timing signal and the acceleration signal during a third predetermined period of time subsequent to the second predetermined period of time whereby if the acceleration signal remains in a predetermined very low range during the third predetermined period of time then it is determined that the user is immobile and a fall detection event is confirmed.

Preferably when a fall condition is determined by the processor a fall signal is transmitted to a remote location.

Preferably when a fall condition is determined by the processor then a fall signal is communicated locally.

Preferably the acceleration signal is referenced against a reference frame.

Preferably the reference frame is the surface upon which a user of the fall detection apparatus is supported.

Preferably the fall detection apparatus is a wrist mounted fall detection apparatus.

In a further broad form of the invention there is provided a detection and communication system which reads vital signs of the body of a user utilising a sensing device and applies algorithms to interpret the vital signs and then send a notification with an escalation process to nominated carers if the user is interpreted as having a fall or fit or seizure.

Preferably the device is a smartwatch or smart phone (for example utilising the iOS, Android or Pebble operating systems).

Preferably doctors or other parties can log in to a secured dashboard and check user data in real time.

Preferably doctors or other parties can analyse the history of the user.

Preferably users/patients can also utilise user data derived by the system to keep track of fall or fit or seizure episodes and monitor their progress.

In yet a further broad form of the invention there is provided a seizure detection apparatus comprising:

an accelerometer which communicates an acceleration signal to a processor;

the acceleration signal quantifying acceleration on a substantially continuous basis relative to a reference frame;

a timer which communicates a time reference signal to the processor;

the processor monitoring the acceleration signal on a substantially continuous basis;

the processor monitoring the timing signal on a substantially continuous basis;

and whereby if the acceleration signal oscillates within a predetermined range for a predetermined period of time then a seizure event is determined and signalled.

Preferably the seizure detection apparatus is wrist mounted seizure detection apparatus.

In yet a further broad form of the invention there is provided a sleepwalk detection apparatus comprising:

an accelerometer which communicates an acceleration signal to a processor;

the acceleration signal quantifying acceleration on a substantially continuous basis relative to a reference frame;

a timer which communicates a time reference signal to the processor;

the processor monitoring the acceleration signal on a substantially continuous basis;

the processor monitoring the timing signal on a substantially continuous basis;

and whereby if the acceleration signal indicates a walking movement during a predetermined period of time which exceeds a minimum walking time and which is determined to be a bed time of the user then a sleepwalk event is determined and signalled.

Preferably the sleepwalk detection apparatus is wrist mounted sleepwalk detection apparatus.

In yet a further broad form of the invention there is provided a method of detecting a fall event comprising:

providing an accelerometer which communicates an acceleration signal to a processor;

the acceleration signal quantifying acceleration on a substantially continuous basis relative to a reference frame;

providing a timer which communicates a time reference signal to the processor;

the processor monitoring the acceleration signal on a substantially continuous basis;

the processor monitoring the timing signal on a substantially continuous basis;

and whereby if the acceleration signal is within a first low acceleration range for a predetermined period of time and is followed by a second high acceleration signal in a second predetermined period of time a fall condition is determined by the processor.

In yet a further broad form of the invention there is provided a method of seizure detection comprising:

providing an accelerometer which communicates an acceleration signal to a processor;

the acceleration signal quantifying acceleration on a substantially continuous basis relative to a reference frame;

providing a timer which communicates a time reference signal to the processor;

the processor monitoring the acceleration signal on a substantially continuous basis;

the processor monitoring the timing signal on a substantially continuous basis;

and whereby if the acceleration signal oscillates within a predetermined range for a predetermined period of time then a seizure event is determined and signalled.

In yet a further broad form of the invention there is provided a method of detecting a sleepwalk event comprising:

providing an accelerometer which communicates an acceleration signal to a processor;

the acceleration signal quantifying acceleration on a substantially continuous basis relative to a reference frame;

providing a timer which communicates a time reference signal to the processor;

the processor monitoring the acceleration signal on a substantially continuous basis;

the processor monitoring the timing signal on a substantially continuous basis;

and whereby if the acceleration signal indicates a walking movement during a predetermined period of time which exceeds a minimum walking time and which is determined to be a bed time of the user then a sleepwalk event is determined and signalled.

### BRIEF DESCRIPTION OF DRAWINGS

Embodiments of the present invention will now be described with reference to the accompanying drawings wherein:

FIG. 1 is a logic flow diagram of an alert system in accordance with an embodiment of the invention;

FIG. 2 is a flow chart of a fall detection algorithm applicable to the system of FIG. 1;

FIG. 3 is a flow chart of a seizure detection algorithm applicable to the system of FIG. 1;

FIG. 4 is a flow chart of a sleep walk detection algorithm applicable to the system of FIG. 1;

FIG. 5 is an electronic block diagram of an implementation of the system of FIG. 1;

FIG. 6 is an electronic block diagram of a further implementation of the system of FIG. 1;

FIG. 7 is an electronic block diagram of yet a further implementation of the system of FIG. 1;

### DETAILED DESCRIPTION OF EMBODIMENTS

Broadly what is disclosed is a device, method and system which, in at least some embodiments, can read the vital signs of the body of a user utilising a sensing device such as a smartwatch or smart phone (for example utilising the iOS, Android or Pebble or Tizen operating systems) and apply algorithms to interpret the vital signs and then send a notification with an escalation process to nominated carers if the patient is interpreted as having a fall or fit or seizure. In at least some embodiments doctors or other parties can log in to a secured dashboard and check a patient data in real time. Also in at least some preferred forms doctors or other parties can analyse the history of the patient.

In at least some embodiments users/patients can also use data to keep track of fall or fit or seizure episodes and monitor their progress.

Embodiments of the invention can be applied for example in situations where the patient/user suffers from a medical condition such as epilepsy and which may predispose the patient/user to falls and related events.

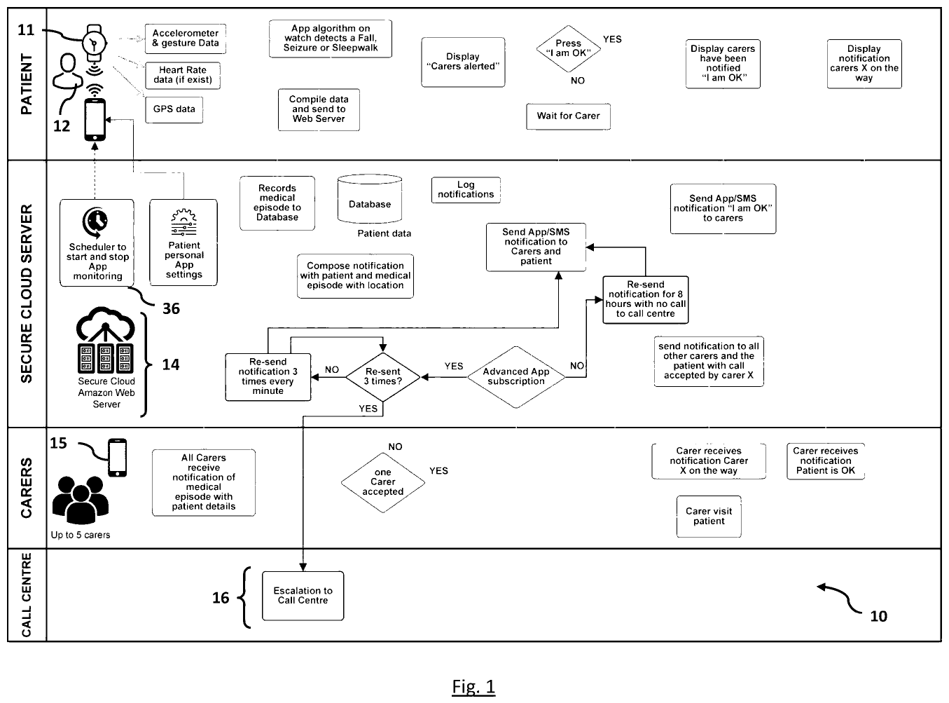

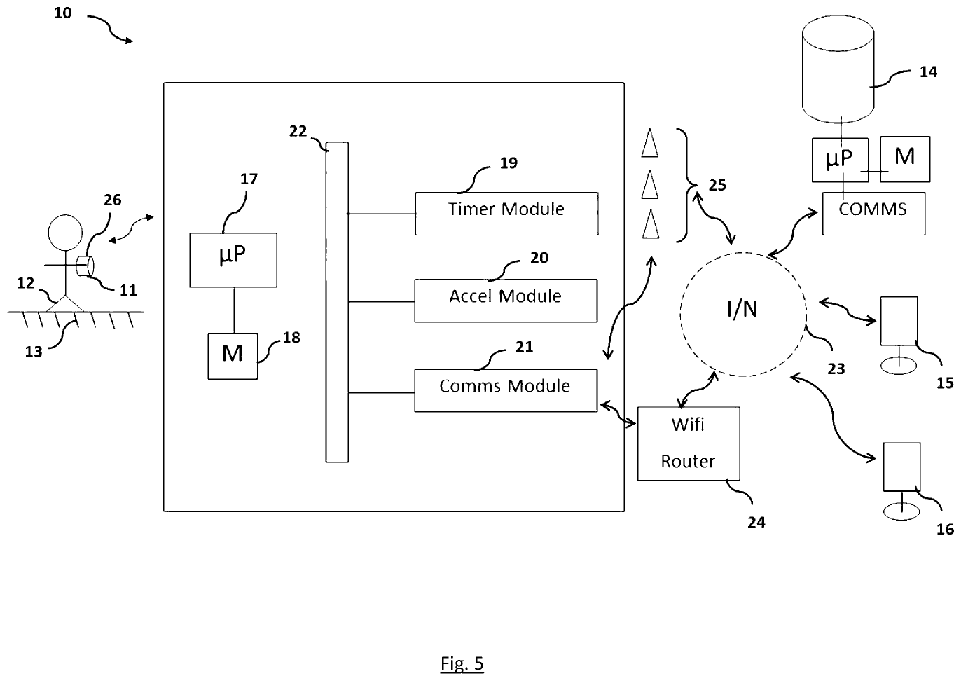

With reference to FIG. 1 and FIG. 5 there is illustrated an alert system 10 in accordance with a first embodiment of the present invention.

In this instance the alert system 10 monitors and analyses data derived from a sensor 11. In preferred forms the sensor 11 is a body worn sensor. In particular forms it may be strapped to the wrist of user 12. In other forms it may be chest mounted, ankle mounted or otherwise, but such that there is a mechanical association as between the sensor 11 and the body of the user 12 sufficient for the sensor to detect parameters associated with the body of the user 12.

Such parameters may include movement of the body relative to a reference frame. In preferred forms the reference frame will be the surface which supports the user 12.

Other parameters may include physiological parameters such as heart rate, ECG waveforms, EEG waveforms, blood pressure, blood glucose, sweat, body temperature and the like.

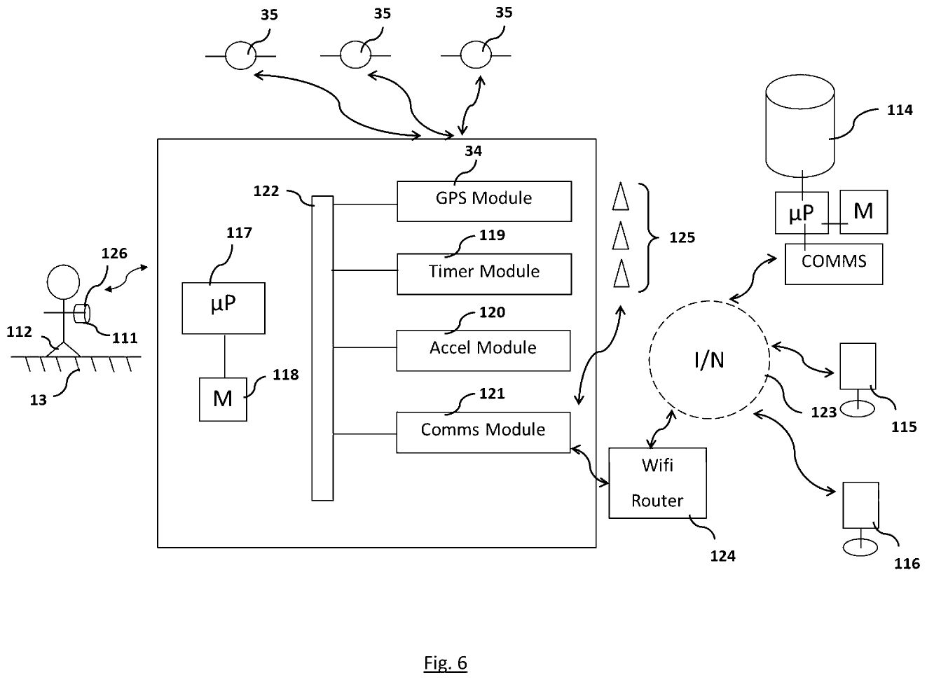

Yet other parameters may include geographic location information and data such as is derived from a GPS module. An embodiment of the device incorporating GPS capability is shown in FIG. 6 wherein like components are numbered as for the first embodiment except in the 100 series. In this instance, in addition to time a module 119, acceleration sensing module 120 and communications module 121 there is included a GPS module 34 in communication with satellites 35 and, optionally with a Wi-Fi signal as may be provided by Wi-Fi router 124.

In some instances the user 12 will be referred to as a patient although there will be contexts in which the alert system 10 is used whereby user 12 will be the subject of monitoring by the system 10 but the description as a “patient” may not be apt.

Broadly the system 10 comprises components which are networked together and which, in most instances, will be geographically separated from each other.

In a particular form the system 10 includes a sensor 11 mechanically associated with user 12 which is in communication with a server 14. In many instances the sensor and/or the server 14 will also be in communication with carer digital communications devices 15 and also, separately, in communication with call centre digital communication devices 16.

In particular preferred forms the sensor 11 is in the form of a wearable device preferably attached to the wrist of user 12.

The sensor 11 incorporates or is in communication locally with a processor 17, a memory 18, a timer module 19, acceleration sensing module 20 and a communications module 21. In a preferred form the components 17, 18, 19, 20, 21 communicate with each other over bus 22.

In a further distributed form at least the acceleration detection module and communications module may communicate via Bluetooth or other short range radio or electromagnetic transmission capability with the other components forming the sensor 11.

In preferred forms the acceleration sensing module 20 is implemented as at least a three axis accelerometer which permits acceleration to be resolved in three orthogonal axes.

The communications module 21 may communicate with the Internet 23 or other wide area network either by way of Wi-Fi router 24 or via cellular telephone network 25 whereby the sensor 11 is placed in data communication with server 14, carer digital communications device 15 and call centre digital communications device 16.

The system 10 further includes a scheduler 36 in a preferred form executed as an application on the server 14. A primary function of the scheduler 36 is to start and stop monitoring effected by the sensor 11.

In a particular form the functionality is to automatically start the monitoring of the application on sensor 11 in the morning and close it at night, for fall and seizure event detection. For sleepwalking event detection it will be started at bed time and closed in the morning.

In use

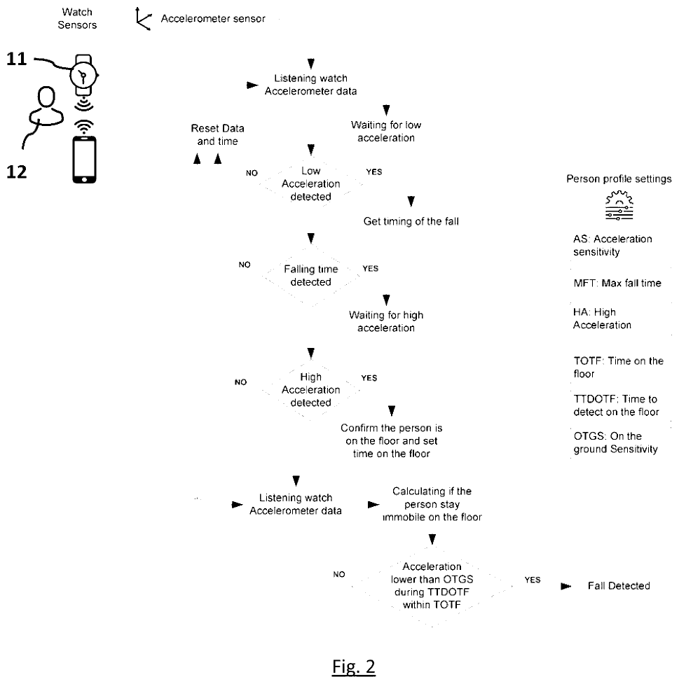

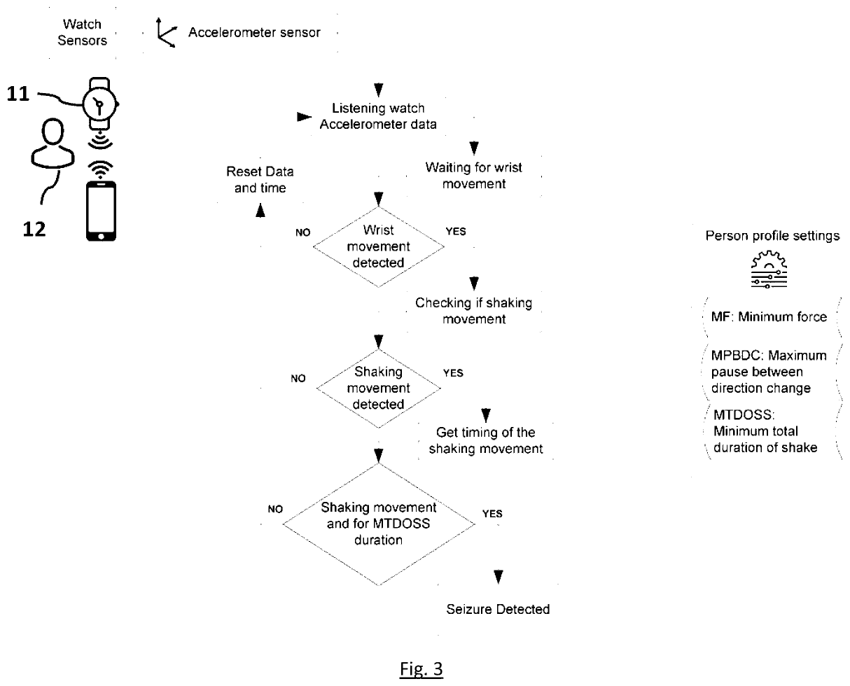

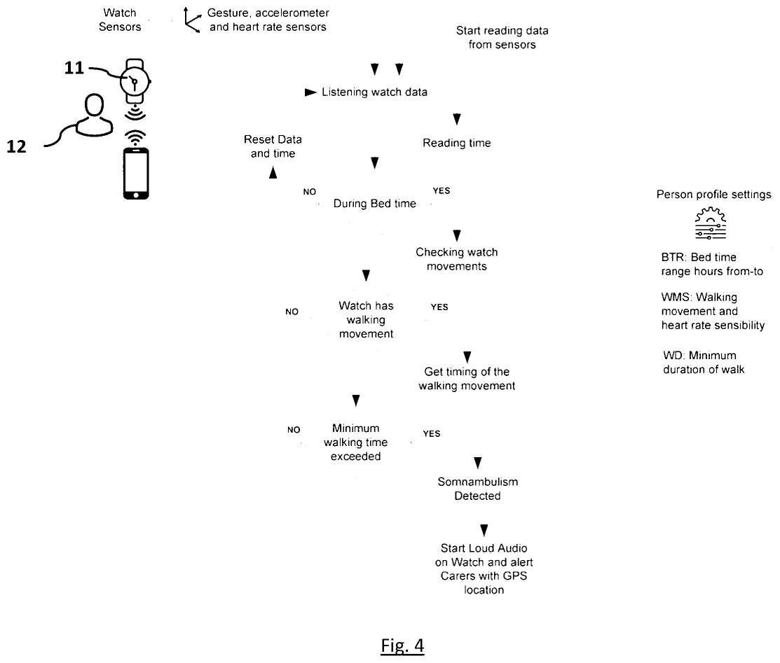

As best seen initially in FIG. 1, the arrangement of FIG. 5 is utilised to monitor at least the accelerometer data and apply an algorithm referenced at least to timing data derived from timer module 19 in order to determine if a fall condition/event has occurred (as outlined in the flowchart of FIG. 2), whether a seizure event has been detected (in accordance with the flowchart of FIG. 3) or whether a sleep walk event has been detected (with reference to the flowchart of FIG. 4).

The event is then communicated to one or more of the server 14, the carer digital communications device 15 and call centre digital communications device 16 in accordance with the flowchart of FIG. 1.

In particular forms the event is also communicated locally to the user 12. In preferred forms the event is communicated locally by way of a display 26 associated with the sensor 11.

In preferred forms the display 26 may be a touch sensitive display(or voice activation, apple siri or ok google assistance) whereby the user may communicate with one or more of the server 14, the carer digital communications device 15 or the call centre digital communications device 16.

### Integrated Sensor and Communications Device

In a particular preferred form the sensor 11, 111, 211 may be implemented as a smartwatch App running on an independent smartwatch which has an integrated sim or esim card, such as the Apple watch Series 3 or the LG Urbane LTE Smartwatches.

### Machine Learning Adaptation

In particular preferred forms an artificial intelligence AI capability may be programmed into memory 18 for execution by processor 17. In an alternative form or in addition, an AI program may be executed on the processor associated with server 14. One particular application of the AI capability is to learn from false positive event determination and false negative event determination in order to statistically improve reliability of detection of an event over time and with particular reference to learned attributes of the data associated with any given user 12.

### Sleep Walking Detection

With reference to FIG. 1 in conjunction with FIG. 4 instructions for an algorithm may be stored in memory 18 and executed by processor 17 operating according to the flowchart of FIG. 4 to detect and communicate and alarm,as appropriate, a sleepwalking event.

### Heart Rate Monitoring Event Detection

In preferred forms the sensor 11 may include ECG monitoring capability whereby heart rate monitoring may provide an alert to patient and carer when an unusual heart rate/beat is recorded.

### Audio Functionality

Audio when an event such as a fall, seizure or sleepwalk is detected to alert people around and emergency services. In preferred fauns this is effected by the sensor emitting an audible sound. In particularly preferred forms the sound is loud enough for surrounding people to hear.

### Sensor Condition Monitoring and Communication

Adding capability on the App to send notification to carers about the App monitoring status (making sure the app is monitoring) as well as the battery level of the watch, so carer can contact the patient if there is any issue of the App monitoring

Integration with Other Systems-Telehealth

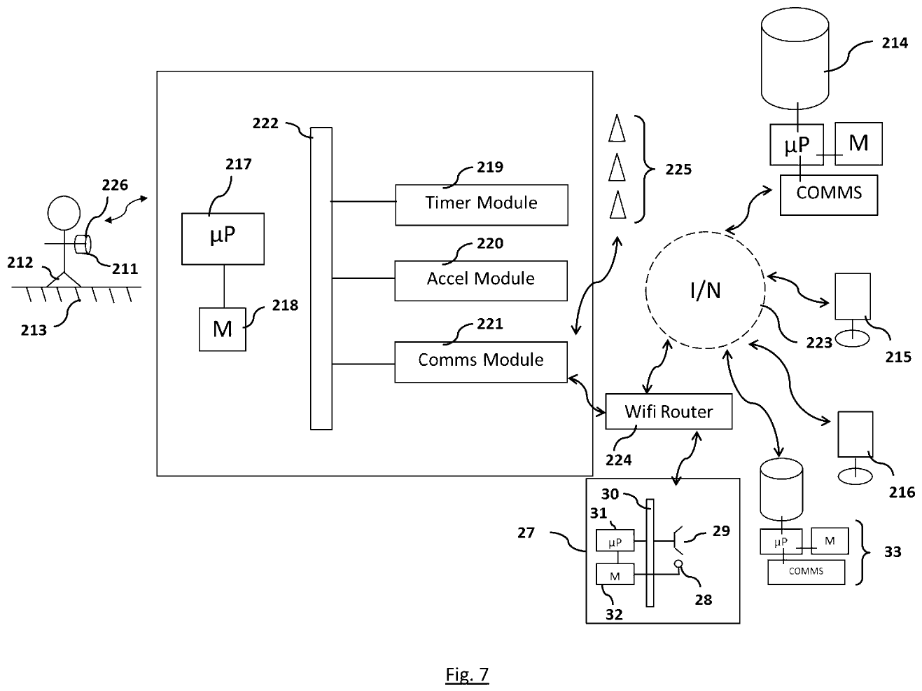

In a particular form and with reference to FIG. 7 wherein like components are numbered as for the first embodiment except in the 200 series, an additional monitoring or sensor device 27 may be located in association with user 12. In preferred forms the additional monitoring or sensor device may be located in the home of the user or the office of the user or other location where the user may spend a predetermined period of time.

The additional monitoring or sensor device 27 includes functionality and communications capability similar to that of sensor 11 but more particularly includes at least microphone 28 and also in preferred forms speaker 29 in communication with a bus 30 which are also in communication with processor 31 and memory 32 and thence in communication with Wi-Fi router 224, Internet 223 and subsequently Web enabled database 33.

In particular forms the additional monitoring or sensor device 27 may take the form of a smart microphone and speaker device of the form currently marketed as the Amazon Echo, or Google home device or the HomePod from Apple.

These devices permit audio pickup typically from an entire room and also audio playback to an entire room. Third-party applications may be run on web enabled server 233 to provide specific functionality to complement the basic functionality which can include voice recognition and giving effect to voice commands by way of communication with other devices located in the vicinity.

In the present instance this arrangement facilitates a telehealth functionality enabling the user at home to talk to carers and emergency workers using at least the voice recognition system built into the additional monitoring or sensing device 27. In a preferred form an application will be loaded onto Web enabled server 33 which, when executed, integrates functionality of the additional monitoring or sensor device 27 with the functionality of the sensor 211.

In a particular form this combining of functionality provides a powerful, integrated body worn sensor with a local room sensor which has at least audio pickup and audio playback capability.

### INDUSTRIAL APPLICABILITY

Embodiments of the present invention have application wherever it is desired to monitor and communicate conditions or events associated with a user.

In particular forms the system has application to fall detection and communication of same to remote locations for the purpose of obtaining assistance or at least monitoring of same.

In at least some embodiments, the system can be applied with advantage to read the vital signs of the body of a user utilising a sensing device such as a smartwatch or smart phone (for example utilising the iOS, Android or Pebble operating systems) and apply algorithms to interpret the vital signs and then send a notification with an escalation process to nominated carers if the patient is interpreted as having a fall or fit or seizure. In at least some embodiments doctors or other parties can log in to a secured dashboard and check a patient data in real time. Also in at least some preferred forms doctors or other parties can analyse the history of the patient.

In at least some embodiments users/patients can also use data to keep track of fall or fit or seizure episodes and monitor their progress.

Embodiments of the invention can be applied for example in situations where the patient/user suffers from a medical condition such as epilepsy and which may predispose the patient/user to falls and related events.

The above describes only some embodiments of the present invention and modifications, obvious to those skilled in the art, can be made thereto without departing from the scope of the present invention.

1. An alert system for communicating an event sensed by a body worn sensor.

2. The system of claim 1 wherein the body worn sensor is mechanically associated with the body.

3. The system of claim 1 or 2 wherein the event is a fall event.

4. The system of any previous claim wherein the sensor includes a processor in communication with memory for on-board processing of at least one signal.

5. The system of any previous claim wherein the sensor includes a timer.

6. The system of any previous claim wherein the sensor includes a GPS device.

7. The system of any previous claim wherein the sensor includes a communications device.

8. The system of any previous claim wherein the communications device includes broadband network interconnectivity for connection to the Internet.

9. The system of any previous claim wherein the communications device includes cellular telephone network interconnectivity for connection of the device to a local cellular telephone network.

10. The system of any previous claim wherein the sensor includes an accelerometer.

11. The system of any previous claim wherein the at least one signal is an acceleration signal.

12. The system of any previous claim wherein the at least one signal is a timing signal.

13. The system of any previous claim wherein the signal is an acceleration signal derived from the accelerometer.

14. The system of any previous claim wherein the signal is a timing signal derived from the timer.

15. The system of any previous claim wherein the signal is a GPS signal derived from the GPS device.

16. The system of any previous claim wherein the event is a fall event.

17. The system of any one of claims 1 to 15 wherein the event is a seizure event.

18. The system of any one of claims 1 to 15 wherein the event is a sleepwalk event.

19. The system of any previous claim further including an additional monitoring or sensing device.

20. The system of claim 19 wherein the additional monitoring or sensing device includes at least a speaker and a microphone and is in communication with a web enabled server.

21. The system of claim 20 wherein the web enabled server executes an application whereby functionality of the body worn sensor is supplemented with the functionality of the additional monitoring or sensing device.

22. The system of claim 21 wherein the body worn sensor is mounted to the wrist of a user.

23. the system of any one of claims 1 to 22 wherein an artificial intelligence AI capability is programmed into memory 18 for execution by processor 1 of the body worn sensor.

24. The system of claim 23 wherein an AI program is executed on the processor associated with server located remote from the sensor 14.

25. The system of claim 23 or 24 wherein the AI capability learns from false positive event determination and false negative event determination in order to statistically improve reliability of detection of an event over time and with particular reference to learned attributes of the data associated with any given user 12.

26. A fall detection apparatus comprising:
an accelerometer which communicates an acceleration signal to a processor;
the acceleration signal quantifying acceleration on a substantially continuous basis relative to a reference frame;
a timer which communicates a time reference signal to the processor;
the processor monitoring the acceleration signal on a substantially continuous basis;
the processor monitoring the timing signal on a substantially continuous basis;
and whereby if the acceleration signal is within a first low acceleration range for a predetermined period of time and is followed by a second high acceleration signal in a second predetermined period of time a fall condition is determined by the processor.

27. The fall detection apparatus of claim 26 wherein the processor monitors the timing signal and the acceleration signal during a third predetermined period of time subsequent to the second predetermined period of time whereby if the acceleration signal remains in a predetermined very low range during the third predetermined period of time then it is determined that the user is immobile and a fall detection event is confirmed.

28. The fall detection apparatus of claim 26 or claim 27 wherein when a fall condition is determined by the processor a fall signal is transmitted to a remote location.

29. The fall detection apparatus of claim 26 or 27 or 28 wherein when a fall condition is determined by the processor then a fall signal is communicated locally.

30. The fall detection apparatus of claim 26, 27, 28 or 29 wherein the acceleration signal is referenced against a reference frame.

31. The fall detection apparatus of claim 30 wherein the reference frame is the surface upon which a user of the fall detection apparatus is supported.

32. The fall detection apparatus of any one of claims 26 to 31 wherein the fall detection apparatus is a wrist mounted fall detection apparatus.

33. A detection and communication system which reads vital signs of the body of a user utilising a sensing device and applies algorithms to interpret the vital signs and then send a notification with an escalation process to nominated carers if the user is interpreted as having a fall or fit or seizure.

34. The system of claim 33 wherein the device is a smartwatch or smart phone (for example utilising the iOS, Android or Pebble operating systems).

35. The system of claim 33 or 34 wherein doctors or other parties can log in to a secured dashboard and check user data in real time.

36. The system of any one of claims 33 to 36 wherein doctors or other parties can analyse the history of the user.

37. The system of any one of claims 33 to 36 wherein users/patients can also utilise user data derived by the system to keep track of fall or fit or seizure episodes and monitor their progress.

38. A seizure detection apparatus comprising:
an accelerometer which communicates an acceleration signal to a processor;
the acceleration signal quantifying acceleration on a substantially continuous basis relative to a reference frame;
a timer which communicates a time reference signal to the processor;
the processor monitoring the acceleration signal on a substantially continuous basis;
the processor monitoring the timing signal on a substantially continuous basis;
and whereby if the acceleration signal oscillates within a predetermined range for a predetermined period of time then a seizure event is determined and signalled.

39. The seizure detection apparatus of claim 38 wherein the seizure detection apparatus is wrist mounted seizure detection apparatus.

40. A sleepwalk detection apparatus comprising:
an accelerometer which communicates an acceleration signal to a processor;
the acceleration signal quantifying acceleration on a substantially continuous basis relative to a reference frame;
a timer which communicates a time reference signal to the processor;
the processor monitoring the acceleration signal on a substantially continuous basis;
the processor monitoring the timing signal on a substantially continuous basis;
and whereby if the acceleration signal indicates a walking movement during a predetermined period of time which exceeds a minimum walking time and which is determined to be a bed time of the user then a sleepwalk event is determined and signalled.

41. The sleepwalk detection apparatus of claim 40 wherein the sleepwalk detection apparatus is wrist mounted sleepwalk detection apparatus.

42. A method of detecting a fall event comprising:
providing an accelerometer which communicates an acceleration signal to a processor;
the acceleration signal quantifying acceleration on a substantially continuous basis relative to a reference frame;
providing a timer which communicates a time reference signal to the processor;
the processor monitoring the acceleration signal on a substantially continuous basis;
the processor monitoring the timing signal on a substantially continuous basis;
and whereby if the acceleration signal is within a first low acceleration range for a predetermined period of time and is followed by a second high acceleration signal in a second predetermined period of time a fall condition is determined by the processor.

43. A method of seizure detection comprising:
providing an accelerometer which communicates an acceleration signal to a processor;
the acceleration signal quantifying acceleration on a substantially continuous basis relative to a reference frame;
providing a timer which communicates a time reference signal to the processor;
the processor monitoring the acceleration signal on a substantially continuous basis;
the processor monitoring the timing signal on a substantially continuous basis;
and whereby if the acceleration signal oscillates within a predetermined range for a predetermined period of time then a seizure event is determined and signalled.

44. A method of detecting a sleepwalk event comprising:
providing an accelerometer which communicates an acceleration signal to a processor;
the acceleration signal quantifying acceleration on a substantially continuous basis relative to a reference frame;
providing a timer which communicates a time reference signal to the processor;
the processor monitoring the acceleration signal on a substantially continuous basis;
the processor monitoring the timing signal on a substantially continuous basis;
and whereby if the acceleration signal indicates a walking movement during a predetermined period of time which exceeds a minimum walking time and which is determined to be a bed time of the user then a sleepwalk event is determined and signalled.
